(19) United States
(12) Patent Application Publication
(10) Pub. No.: US 2018/0280429 A1
(43) Pub. Date: Oct. 4, 2018

(54) APPLICATION OF NITRATE IN PREPARATION OF DRUG PREVENTING OR TREATING BONE METABOLIC DISEASES

(71) Applicant: Capital Medical University, Beijing (CN)

(72) Inventors: Songling Wang, Beijing (CN); Baoxing Pang, Beijing (CN); Xingmin Qu, Beijing (CN)

(21) Appl. No.: 15/764,877

(22) PCT Filed: Dec. 22, 2015
(86) PCT No.: PCT/CN2015/098314

§ 371 Date: Mar. 29, 2018

(30) Foreign Application Priority Data
Sep. 30, 2015 (CN) 201510634301.4

(51) Int. Cl. A61K 33/00; A61P 19/10

(52) U.S. Cl. CPC A61K 33/00; A61P 19/10

(57) ABSTRACT

A drug preventing or treating bone metabolic diseases and containing a nitrate, and applications of a nitrate in the preparation of a drug preventing or treating bone metabolic diseases, and the preparation of drugs regulating bodily immune abnormalities and bone marrow mesenchymal stem cell abnormalities.

### TECHNICAL FIELD

The present application relates to the field of medicine, and more specifically, relates to applications of a nitrate in the preparation of a drug preventing or treating bone metabolic diseases, and the preparation of drugs regulating bodily immune abnormalities and bone marrow mesenchymal stem cell abnormalities.

### BACKGROUND

Nitrate is an inorganic substance widely found in nature. Plants can absorb and use nitrate in soil to supply the nitrogen needed in the body. Most of the nitrate in food comes from green vegetables. The traditional view is that dietary nitrates and nitrites have a potential carcinogenic tendency. Then it is recognized that nitrite will only show a carcinogenic effect when it is combined with amines to form nitrite amines under certain conditions and reaches a certain concentration. At present, the exact role of oral nitrate in enhancing blood perfusion and regulating energy metabolism has reached a consensus, which laid a foundation for the application of bone metabolism diseases.

Bone metabolic diseases refer to the bone disease that resulted from bone biochemical metabolic disorders due to the innate or acquired factors of the body destroying or interfering with the normal bone metabolism and biochemical status. Abnormal bone density disease, one of the common types, refers to a systemic bone disease caused by direct or indirect causes, but it may also be prominently reflected as a bone change in a part of the body, such as the general body decalcification caused by hyperparathyroidism and local bone resorption caused by malignant tumor invasion of bone tissue and so on. Abnormal bone density disease is mainly characterized by a disorder or abnormality in conversion between bone formation and bone resorption, such as the bone formation or bone resorption may be reduced or increased, the bone matrix formation may be absent or increased, the mineralization may also be lacking, insufficient, or excessively deposited. As a result, bones may show looseness, softening, hardening, or excessive calcification, or two or more of the above manifestations. Among them, osteoporosis is the most common degenerative bone density disease in the elderly, especially in postmenopausal women, with reduction of systemic bone mass, degeneration of bone tissue microstructure, decreased bone strength, increased bone fragility, easy to fracture and body pain as the main lesion characteristics. Currently, drugs for the treatment of osteoporosis in the clinic can be divided into drugs for anti-bone resorption, drugs for promoting bone-forming, and drugs promoting bone mineralization and so on. The most commonly used is estrogen replacement therapy (ERT), whose efficacy has been fully affirmed. However, a long-term use of estrogen increases the risk of endometrial cancer, breast cancer, and cardiovascular disease.

At present, there is no research or patent reporting the application of nitrate in the prevention and treatment of bone metabolic diseases, especially bone density abnormalities.

### SUMMARY

The object of the present application is to provide an application of a nitrate in the preparation of a drug preventing or treating bone metabolic diseases, and a drug containing the nitrate for preventing or treating bone metabolic diseases, aiming at the defects of poor efficacy of the existing therapeutic drugs for bone metabolism diseases.

In one aspect, the technical solution adopted by the present application to solve its technical problems is to provide an application of a nitrate in the preparation of a drug preventing or treating bone metabolic diseases. This new use of nitrates is first discovered and provided by the present application.

In a preferred embodiment of the nitrate application according to the present application, the nitrate is configured for preparing drug preventing or treating osteoporosis. The osteoporosis includes primary osteoporosis such as postmenopausal or senile osteoporosis, and secondary osteoporosis caused by various endocrine disorders or abolishment. By a large number of experiments, the present application proves that the addition of nitrate to the drinking water of ovariectomized rats can significantly relieve the loss of bone mass in rats. The nitrate has no obvious adverse reactions at the time of preservation of bone mass, and has no significant influence on the body weight of rats. Based on the same principle, the nitrate-produced medicament of the present application is also suitable for diseases in which bone loss is required to be alleviated. Further research shows that nitrate can also regulate bodily immune abnormalities and bone marrow mesenchymal stem cell abnormalities, therefore, the novel use of nitrates in the treatment of osteoporosis discovered for the first time by the present application may be achieved through nitrate regulating immune immunity and bone marrow mesenchymal stem cells.

In a preferred embodiment of the nitrate application according to the present application, the nitrate is further configured for preparing a drug preventing or treating abnormal bone density diseases. These abnormal bone density diseases are caused by that congenital or acquired factors disrupt or interfere with normal bone metabolism and biochemical status, leading to a break in the balance of osteogenesis and osteoclasts, resulting in decreased bone density and even loss of bone mass. It mainly includes osteolytic changes caused by malignant tumors invading bone tissue, loss of bone mass caused by rheumatoid arthritis in violation of cartilage and bone tissue, and loss of bone mass due to thyroid dysfunction and so on. As the present application has proved through experiments that nitrate can significantly relieve the loss of bone mass in rats, therefore, it can also be used to treat these abnormal bone density diseases, especially abnormal bone density caused by bodily immune abnormalities and/or bone marrow mesenchymal stem cell abnormalities.

In a preferred embodiment of the nitrate application according to the present application, the nitrate is a salt composed of a metal ion and a nitrate ion. In the present application, the nitrate ion is the main active ingredient of the nitrate. Therefore, any pharmaceutically acceptable salt containing nitrate can be used, preferably a salt composed of a metal ion and a nitrate ion, or ammonium nitrate and the like. Therefore, the nitrate of the present application is selected from a group consisting of one or more of the followings: sodium nitrate, potassium nitrate, and calcium nitrate and ammonium nitrate and so on.

In a preferred embodiment of the nitrate application according to the present application, the nitrate is provided in the form of a food or an extract that is rich in nitrate. The nitrate in the present application can be provided not only as a compound but also can be provided in a food or an extract. Therefore, the present application also provides new uses for these nitrate-rich foods or extracts for the preparation of a drug preventing or treating bone metabolic diseases accordingly. For example, the concentration of nitrate in vegetables such as spinach, beetroot, white radish, and celery can reach more than 3000 mg/Kg, which is several to ten times as high as that of other common foods.

In a preferred embodiment of the nitrate application according to the present application, a daily dose of the nitrate is 0.1-0.5 mmol/Kg b.w. More preferably, a daily dose of the nitrate is 0.5 mmol/Kg b.w.

In another aspect, a drug preventing or treating bone metabolic diseases is also provided by the present application, which contains nitrate as an active ingredient. Preferably, a drug preventing or treating abnormal bone density diseases is provided, in which the nitrate is contained as the active ingredient. More preferably, a drug preventing or treating osteoporosis is provided, in which the nitrate is contained as the active ingredient. The nitrates may also be provided in various forms and dosages as described previously.

In a preferred embodiment of the present application, dosage forms of the drug preventing or treating bone metabolic diseases are tablets, capsules, pills, injections, syrups, oral liquids, granules or patches. Among them, the nitrate oral solution can provide a more convenient and effective administration route, and the concentration of the nitrate oral solution is preferably 0.5 mmol/L-2.5 mmol/L, more preferably 2.5 mmol/L.

In a further aspect, an application of nitrate in preparing drug regulating bodily immune abnormalities is further provided by the present application, and more preferably, nitrate plays a very good regulatory role in immunologically abnormal diseases in which regulatory T lymphocytes are downregulated and interleukin 17 is upregulated. The present application uses flow cytometry and other technical methods to detect the immune regulation of ovariectomized rats, and finds that nitrate can regulate the immunobiological behavior of ovariectomized rats, such as, relieving the decline of regulating T lymphocytes (Treg) of the ovariectomized rats, relieving the down-regulation of TGF-β1 in ovariectomized rats, and relieving the up-regulation of IFN-γ and IL-17. At the same time, a drug regulating bodily immune abnormalities is also provided by the present application, in which the nitrate is contained as the active ingredient. The nitrates may also be provided in various forms and dosages as described previously.

In another further aspect, an application of nitrate in the preparation of drug regulating bone marrow mesenchymal stem cell abnormalities is also provided by the present application. More preferably, nitrate plays a very good regulatory role in diseases in which bone marrow mesenchymal stem cells have reduced osteogenic potential. In the present application, the stem cell activity of ovariectomized rats is detected by a technical method such as stem cell culture, and it is found that nitrate can regulate the biological behavior of bone marrow mesenchymal stem cells in ovariectomized rats, for example, significantly reducing the BMMSCs proliferation ability of ovariectomized rats, significantly enhancing its osteogenic differentiation. At the same time, a drug regulating bone marrow mesenchymal stem cell abnormalities is also provided by the present application, in which the nitrate is contained as the active ingredient. The nitrates may also be provided in various forms and dosages as described previously.

The present application provides new uses of nitrates in the preparation of drugs preventing or treating bone metabolic diseases. Moreover, through a large number of experiments, it has been proved that nitrate can significantly relieve the loss of bone mass in rats. The nitrate has no obvious adverse reactions at the time of bone preservation, and has no significant influence on the body weight of rats. In addition, immune and bone marrow mesenchymal stem cell biological behavior can be controlled in ovariectomized rats. Therefore, it can be prepared as drugs and used for the prevention and/or treatment of osteoporosis and other bone density abnormalities, and bodily immune abnormalities and bone marrow mesenchymal stem cell abnormalities.

### BRIEF DESCRIPTION OF THE DRAWINGS

The present application will be further described below with reference to the accompanying drawings and embodiments. In the drawings:

FIGS. 1A-1F are graphs illustrating the effects of nitrate on bone density and trabecular bone in ovariectomized rats.

FIGS. 2A and 2B are graphs illustrating the effects of nitrate on tibia weight and body weight in ovariectomized rats, respectively.

FIGS. 3A and 3B are graphs illustrating the difference of nitrate content and nitrite content in serum after drinking water containing nitrate for 3 months, respectively.

FIGS. 4A-4D are graphs illustrating the effects of nitrate on immune regulation in ovariectomized rats.

FIGS. 5A and 5B are graphs illustrating the results of the nitrate in the improvement of BMMSC defects in ovariectomized rats.

### DETAILED DESCRIPTION OF THE PREFERRED EMBODIMENT

In order to make the purpose, technical solutions and advantages of the present application clearer, the present application will be further described in detail with reference to the accompanying drawings and embodiments.

1. Establishment of Osteoporosis Model and Nitrate Administration

12-week-old female SD rats were selected for the osteoporosis model and bilateral ovaries of them were removed. The experiment objects were divided into 3 groups: the sham operation group (Sham), the ovariectomized group (OVX) and the nitrate group (Nitrate), and there were 10 rats in each group. In the nitrate group, drinking water with sodium nitrate was given on the second day after ovariectomy (ovarianectomy) at a concentration of 2.5 mmol/L, and the daily intake of nitrate in each rat was about 0.5 mmol/Kg bw, the water with nitrate was given for 12 weeks, while normal drinking water was given in the other groups.

2. Bone Density Detection

The rat tibia was completely separated, and all attached muscles and connective tissue were removed. It was wrapped with wet gauze soaked in physiological saline and stored at −20° C. The upper part of the tibia was scanned with a Skyscan 1162 Micro-CT. The scan thickness was 9 μm. The bone density and trabecular structure were quantitatively analyzed using the attached software.

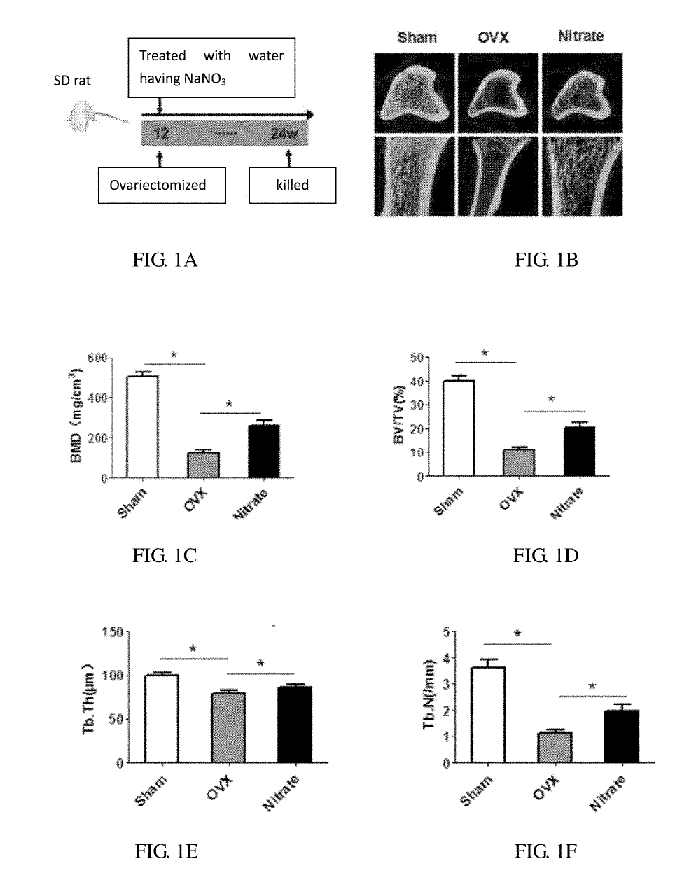

Referring to FIGS. 1A-1F, which are graphs illustrating the effects of nitrate on bone density and trabecular bone in ovariectomized rats. Wherein, FIG. 1A is a schematic diagram of an experimental process of the nitrate group, in which a 12-week-old SD rat undergoing an ovariectomy operation was given drinking water with sodium nitrate, and after 12 weeks, the animals were sacrificed to collect specimens. FIG. 1B shows the MicroCT of the upper tibia of the rat, which shows that the tibial bone mass of the ovariectomized group (OVX) was significantly lost and after giving the nitrate, the bone mass of the tibia lost in the ovariectomized rats was significantly relieved. FIGS. 1C-1F are graphs of bone density (BMD), bone volume fraction (BV/TV), trabecular thickness (Tb.Th), and trabecular number (Tb.N), respectively, of MicroCT quantitative analysis (*P<0.05) for each group. The results show that the above parameters of the ovariectomized group (OVX) decreased significantly, while the Nitrate group was able to partially recover the above indicators.

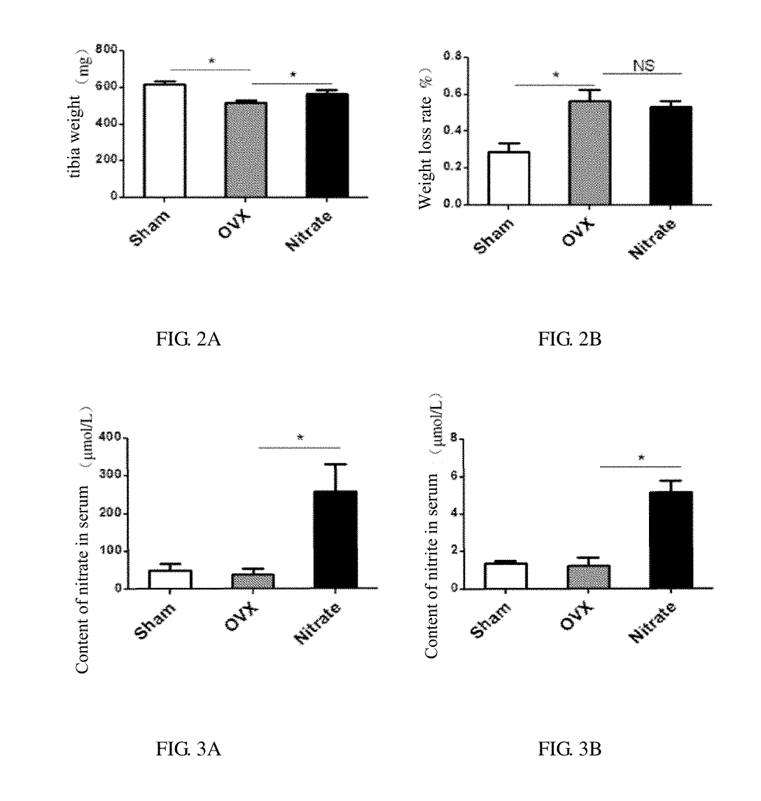

FIGS. 2A and 2B are graphs illustrating the effects of nitrate on tibia weight and body weight in ovariectomized rats, respectively, (*P<0.05). The results show that the drinking water with nitrate can relieve the decrease of tibia weight in ovariectomized rats and has no significant effect on the weight gain of ovariectomized rats.

3. Measurement of Nitrate and Nitrite in Serum

(1) Preparation of the standard: the double dilution of the original standard was 200 μM-3.125 μM

(2) Determination of Nitrite Concentration:

1) Adding 50 μl reaction buffer to blank holes;

2) Adding 50 μl sample or standard to reaction buffer;

3) Adding 50 μl of reaction buffer to each hole in sequence;

4) Adding 50 μl of Griess Reagent I in each hole in sequence;

5) Adding 50 μl of Griess Reagent II to each hole and incubating at room temperature for 10 minutes;

6) Reading the OD value at 540 nm with a microplate reader and the value at 690 nm is used as calibration.

(3) Determination of Nitrate Concentration:

1) Adding 50 μl reaction buffer to blank holes;

2) Adding 50 μl sample or standard to reaction buffer;

3) Adding 25 μl of nicotinamide adenine dinucleotide (NADH) to each hole in sequence;

4) Adding 25 μl of nitrate reductase to each hole in sequence;

5) Adding 50 μl of Griess Reagent I to each hole in sequence;

6) Adding 50 μl of Griess Reagent II to each hole in sequence and incubating at room temperature for 10 minutes;

7) Reading the OD value at 540 nm with a microplate reader and the value at 690 nm is used as calibration.

Referring to FIGS. 3A and 3B, which are graphs (*p<0.05) illustrating the difference of nitrate content and nitrite content in serum after drinking water containing nitrate for 3 months, respectively. Wherein, the content of nitrate and the content of nitrite in serum were significantly increased by drinking water with nitrate.

4. Detection of Immune Indicators

(1) The Detection of the Peripheral Blood Regulatory T Cell Ratio in the Rats

1) Blood was collected from the abdominal aorta of the rats in each group and the serum was collected.

2) 100 μl 0.1% BSA is added for resuspending, and then Anti-CD4, Anti-CD25 are added for incubating on ice for 30 minutes while avoiding light.

3) The obtained mixture is centrifuged at 1500 rpm for 10 minutes, and then the supernatant is removed and 0.1% BSA is added for resuspending.

4) The membrane-permeation solution is added on 4° C., 10 minutes.

5) The obtained mixture is centrifuged at 1500 rpm for 10 minutes, and then the supernatant is removed and 0.1% BSA is added for resuspending.

6) Anti-Foxp3 is added for incubating on ice for 30 minutes while avoiding light.

7) The obtained mixture is centrifuged, and then the supernatant is removed and 500 μl 0.1% BSA is added for resuspending.

8) Detect by the flow cytometry.

(2) Detection of TGF-β1, IFN-γ and IL-17

1) The serum is diluted for 10 times, and 0.1 ml of which is placed into the 96-well plate of the kit. At the same time, the standard in the kit is taken and diluted in proportion, and then placed into the 96-well plate and incubated at 37° C./at room temperature for 30 minutes/1 hour according to the instructions. The obtained object is washed with a buffer for 3 times and each time lasts for 3 minutes.

2) 100 μl enzyme-labeled antibody is added for incubating for 15 minutes/30 minutes at 37° C./at room temperature. The obtained object is washed for 3 times and each time lasts for 3 minutes.

3) 100 μl substrate solution is added and then incubated for 15 minutes at room temperature/37° C.

4) 100 μl stop solution is added to stop the reaction for 5 minutes.

5) The readings of the enzyme labeled instrument at 450 nm is obtained.

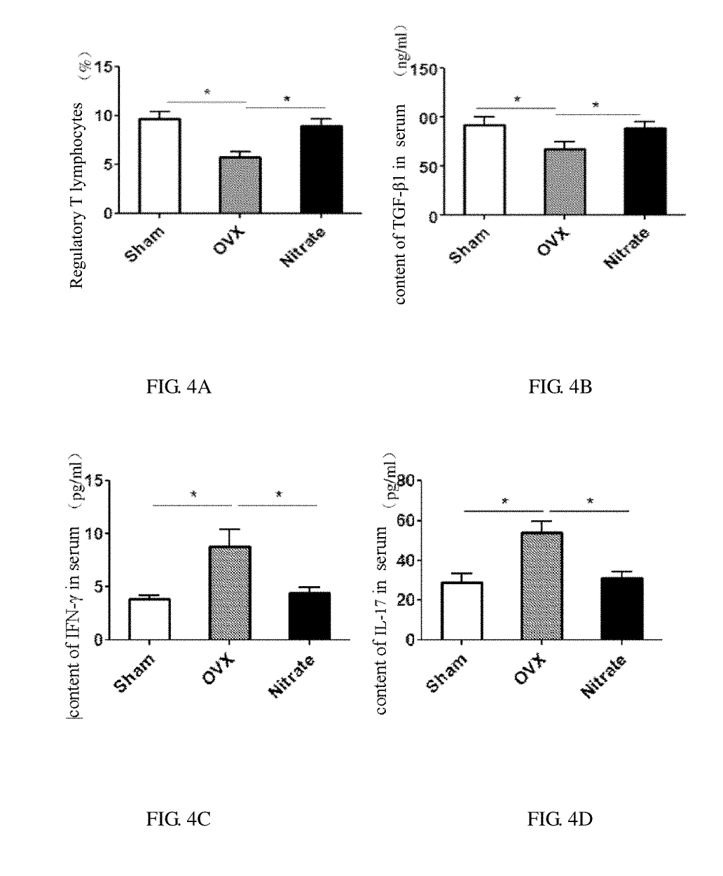

FIGS. 4A-4D are graphs illustrating the effects of nitrate on immune regulation in ovariectomized rats (*P<0.05). FIG. 4A shows the proportion of regulatory T lymphocytes in peripheral blood CD4 positive cells obtained by flow cytometry analysis of rat peripheral blood from each group. As shown in the Figure, drinking water with nitrate can reduce the decline of the regulatory T lymphocytes (Treg) of the ovariectomized rat. FIGS. 4B-4D show the levels of TGF-β1, IFN-γ and IL-17 in the serum of each group. The results show that drinking water with nitrate could alleviate the down-regulation of TGF-β1 in ovariectomized rats and alleviate the up-regulation of IFN-γ and IL-17.

5. Stem Cell Proliferation and Osteogenic Differentiation

(1) Culture of Rat BMMSC

After necropsy, the skin of the hind limbs was removed. After exposing the femur and tibia, the bone marrow was extracted with a 1 mL syringe to flush out from the stump. Cells were cultured in the α-MEM complete medium (containing 10% of bovine serum), and subcultured after the cells were clonally grown until 80% of the fusion.

(2) CCK8 Method

100 μl of cell suspension was inoculated in the 96-well plate, and the plate was pre-cultured in the incubator for 24 hours (under the condition of 37° C., 5% CO₂). Then the plate was incubated in an incubator for 72 hours. 100 μl of culture medium was added to each well and 10 μl of CCK-8 solution was added to each hole. After 2 hours, the absorbance at 450 nm was measured with an enzyme labeled instrument.

(3) Osteogenic Differentiation

Preparation of osteoinductive medium: adding 2 mmol/L glutamine, 100 U/ml penicillin and 100 μg/ml streptomycin, 10 mM β-glycerophosphate sodium, and 10 nM Dexamethasone and 50 mg/L vitamin C to 10% fetal bovine serum α-MEM medium. The 3-4th generation cells were inoculated into a 6-well plate at a concentration of 2×10³/cm², and after the cells were grown to 80% confluent, it was replaced with the osteoinductive medium, and the medium was changed every 2 days. The formation of calcium nodules was observed under light microscope. After 2 weeks of induction, alizarin red staining was performed.

4) Alizarin Red Staining

1) Removing the medium and washing it twice with PBS;

2) Fixing it with 70% ethanol at 4° C. for 1 h;

3) Washing it with double distilled water for 2 times;

4) Staining it with 40 mM alizarin red solution (pH 4.2) at room temperature for 1-10 minutes and visually observing the coloration;

5) Washing it with double distilled water for 5 times and gently blowing;

6) Observing it under the microscope and collecting images.

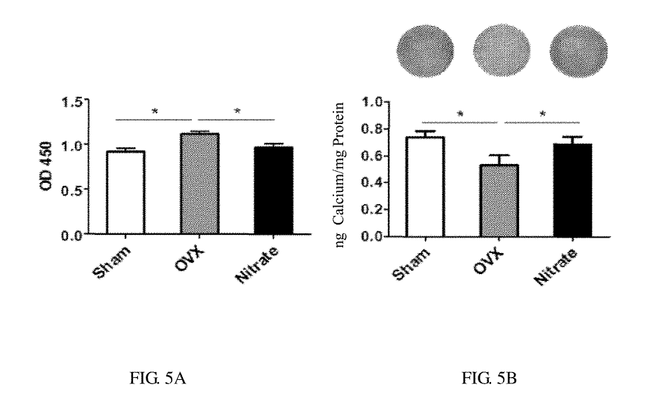

Referring to FIGS. 5A and 5B, which are graphs illustrating the results of the nitrate in the improvement of BMMSC defects in ovariectomized rats (*P<0.05). FIG. 5A shows the OD450 value detected by CCK8 of the third generation cells which are obtained by primary culture of the BMMSC of the rats of each group according to the above method. The results show that the drinking water with nitrate significantly reduced the proliferation of the BMMSCs of ovariectomized rats. FIG. 5B shows the results of alizarin red staining and quantification after 21 days of osteogenesis induction of the BMMSC of rats in each group according to the above method. The results show that the drinking water with nitrate significantly enhanced osteogenic differentiation of the BMMSCs of ovariectomized rats.

6. Statistical Method

SPSS 17.0 statistical software was used for statistical analysis. Multiple sets of measurement data were compared using ANOVA analysis. P<0.05 was statistically significant.

The present application has been described based on specific embodiments, but those skilled in the art should understand that various changes and equivalent substitutions can be made without departing from the scope of the present application. In addition, in order to adapt to the specific occasions or materials of the present application, the present application is subject to numerous modifications without departing from the scope of protection thereof. Therefore, the present application is not limited to the specific embodiments disclosed herein, but includes all embodiments that fall within the scope of the claims.

1-10. (canceled)

11. An application of nitrate in a preparation of a drug preventing or treating bone metabolic diseases.

12. The application according to claim 11, wherein the nitrate is used for preparing a drug preventing or treating abnormal bone density diseases.

13. The application according to claim 11, wherein the nitrate is used for preparing a drug preventing or treating osteoporosis.

14. The application according to claim 11, wherein the nitrate is selected from a group consisting of one or more of the followings: sodium nitrate, potassium nitrate, and calcium nitrate and ammonium nitrate.

15. The application according to claim 12, wherein the nitrate is selected from a group consisting of one or more of the followings: sodium nitrate, potassium nitrate, and calcium nitrate and ammonium nitrate.

16. The application according to claim 13, wherein the nitrate is selected from a group consisting of one or more of the followings: sodium nitrate, potassium nitrate, and calcium nitrate and ammonium nitrate.

17. The application according to claim 14, wherein, the nitrate is provided in the form of a food or an extract that is rich in nitrate.

18. The application according to claim 15, wherein, the nitrate is provided in the form of a food or an extract that is rich in nitrate.

19. The application according to claim 16, wherein, the nitrate is provided in the form of a food or an extract that is rich in nitrate.

20. The application according to claim 11, wherein, a daily dose of the nitrate is 0.1-0.5 mmol/Kg b.w.

21. The application according to claim 12, wherein, a daily dose of the nitrate is 0.1-0.5 mmol/Kg b.w.

22. A drug preventing or treating bone metabolic diseases comprising nitrate as an active ingredient.

23. The drug preventing or treating bone metabolic diseases according to claim 22, wherein dosage forms of the drug preventing or treating bone metabolic diseases are tablets, capsules, pills, injections, syrups, oral liquids, granules or patches.

24. An application of nitrate in preparing drug regulating system immune abnormalities or bone marrow mesenchymal stem cell abnormalities diseases.
